(19) United States
(12) Patent Application Publication
(10) Pub. No.: US 2014/0212843 A1
(43) Pub. Date: Jul. 31, 2014

(54) Custom Dental Implant Fixture

(71) Applicant: Frank Chiu, Flushing, NY (US)

(72) Inventor: Frank Chiu, Flushing, NY (US)

(21) Appl. No.: 13/753,538

(22) Filed: Jan. 30, 2013

(51) Int. Cl. A61C 8/00; A61K 6/04; A61K 6/02; A61C 5/10

(52) U.S. Cl. CPC A61C 8/0001; A61C 8/005; A61C 8/008; A61C 8/0089; A61C 5/10; A61C 8/0009; A61K 6/04; A61C 8/0037; A61K 6/0205; A61C 8/0022
USPC 433/174; 433/201.1

(57) ABSTRACT

The present invention relates to dental implants, and more particularly to custom dental implant fixture that shortens the extraction to completed restoration period from 6-8 months to 4 months. The invention is to use the existing extraction site as is to place custom implant fixture, thereby avoiding a lengthy extraction healing stage and the subsequent osteotomy surgery.

### REFERENCE TO RELATED APPLICATIONS

This patent application claims the benefits of U.S. Provisional Application No. 61/593,272 filed on Jan. 31, 2012, the disclosure of which is incorporated herein by reference in its entirety.

### BACKGROUND OF THE INVENTION

1. Field of the Invention

The present invention relates to dental implants, and more particularly to custom dental implant fixture that shortens the extraction to completed restoration period from 6-8 months to 4 months. By fabricate and place a custom implant fixture within one week of extraction.

2. Description of Related Art

Currently, dental implantation for a single tooth implant typically consists of a titanium standardize sized, screw type cylindrical implant fixture, an abutment screwed onto the fixture and a crown (either screw retained or cement-able). Due to this standardize sized, screw fixture feature, surgery for implant placement is required: An osteotomy is performed using a series of drills to enlarge the implant site into the bone with cylindrical shape and size similar to implant fixture so implant fixture can be screwed into the bone. The typical time required to extract a tooth, restore it by an implant supported crown is between 6-8 months. This period consists of a minimum of three months to allow for an extraction site to heal, fill in with bone so that the osteotomy can be performed. A further three months is needed to allow the titanium implant fixture to osteointegrate with the bone. Once the implant fixture is integrated, dentist can take an impression of the fixture or abutment and have the lab fabricate the implant crown (in other cases also the custom abutment), which will be delivered around 6-8 months (depending on implant site and initial implant placement stability) from initial tooth extraction. Typically, osteotomy and dental implant placement surgery is performed by oral surgeon or general dentist who completed advanced training in implant surgery. Cost of advance training and equipment needed to do osteotomy and implant placement surgery is estimated around $20,000 to $30,000.

Therefore, there exists a need for a new dental implant fixture system and method that shortens the extraction to completed restoration period and lowers the costs of implant placement surgery.

### SUMMARY OF THE INVENTION

A primary object of the invention is to shortens the extraction to completed restoration period from 6-8 months to 4 months.

Another object of the invention is to use the existing extraction site as is to place implant fixture, thereby avoiding a lengthy extraction healing stage and the subsequent osteotomy surgery.

Yet another object of the invention is to provide a custom implant fixture that is based on the root anatomy, soft tissue of the tooth, tooth location, adjacent teeth location/position, and opposing teeth which replaces traditional standardize sized cylindrical implant fixture.

In an exemplary embodiment of the present invention, there is disclosed a system and method that designs, fabricates and utilizes a Custom Implant Fixture (“CIF” hereinafter) system and abutment to shorten the extraction to completed restoration period from 6-8 months to 4 months. This is possible because the CIF system uses the existing extraction site as is to place custom implant fixture, thereby avoiding a lengthy extraction healing stage and the subsequent osteotomy surgery. The CIF is designed and fabricated based on the impression of the tooth extraction socket site captured via a special 3D dental scanner system into a digital 3D dental CAD software. The CIF is designed as an exact negative of the bone socket site so it can be inserted into the bone socket site without needing to drill a hole (osteotomy). The CIF has most of the same features as the traditional cylindrical fixture. It has an apical end and an abutment end. An internal threaded hole is open to the abutment end for receiving the abutment screw.

When a tooth is extracted a temporary space maintainer is placed inside the bone socket site to maintain the space. An impression is taken in order to design and fabricate a CIF. The patient is released for one week. One week later the CIF is delivered to the dentist from the lab. The temporary space retainer is removed, the socket site is cleaned. The custom implant fixture is inserted in the bone socket site to wait for biointegration with the bone for three months. After three months the abutment is torqued onto the custom implant fixture. Another impression is taken for making a crown. Patient can come back two weeks later for receiving the crown. The entire course takes about 3 and ¾ months, equivalent to 15 weeks. The CIF system shortens about three month from extraction to completed restoration of the tooth when compared with using the standard implant fixture. CIF system drastically reduces the cost of advance training and equipment needed for dental implant placement from $20,000-$30,000 to $2,000 (10% of traditional advance training and equipment).

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is an illustration of the Current Dental Implant Protocol.

FIG. 2 is an illustration of the Custom Implant Fixture Protocol (CIF).

FIG. 3 is an illustration showing the to removal of the intra-root bone.

FIG. 4 is an illustration showing the a socket extended for a perio involved tooth.

FIG. 5 is an illustration showing the geometrical difference between custom implant fixtures (CIF) and the traditional implant fixtures.

### DESCRIPTION OF THE PREFERRED EMBODIMENT

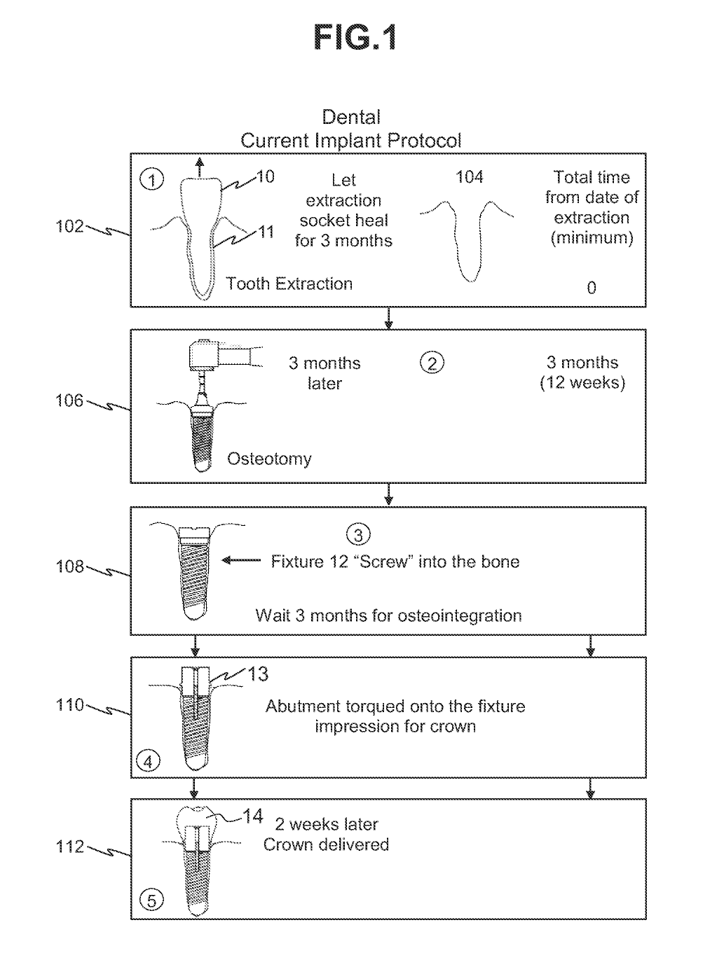

Referring to FIG. 1, there is disclosed a current dental implant protocol. When a tooth 10 is extracted 102, the extraction socket 11 is given 3 months to heal or filled in with bone 104, then an osteotomy is performed 106 and a titanium implant fixture 12 is screwed into the bone 108, another three months is given to allow the titanium implant fixture 12 to osteointegrate with the bone before an abutment 13 is torqued onto the fixture 110. Following the abutment 13 being placed, an impression is taken and sent to lab for fabricating the crown 14. Two weeks later, the crown delivered from the lab to the dentist and put onto the abutment 112. The entire course from tooth extraction to the completed tooth restoration takes about six and half months equivalent to 26 weeks.

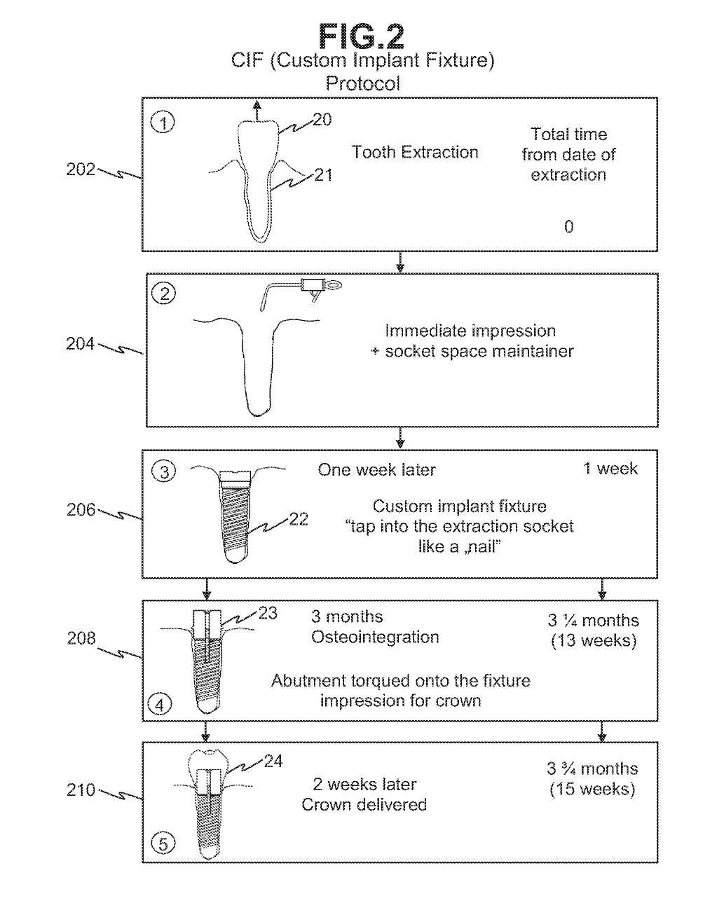

Referring to FIG. 2, there is disclosed a dental implant protocol using a custom implant fixture system and method according to the present invention. The protocol starts from step 202 where a tooth 20 is extracted, an immediate impression is taken to capture the following: the extraction socket 21, teeth adjacent to the extracted tooth; soft tissue and the opposing dentition at step 204. Following taking impression, the extraction/socket site 21 is filled with a solid socket space maintainer that does not reabsorb, or change shape to prevent the bone from filling in before implant fixture placement. The patient is dismissed with a one week appointment. The impression is sent to a lab along with the extracted tooth 20 for designing and fabricating a custom implant fixture 22 and abutment 23.

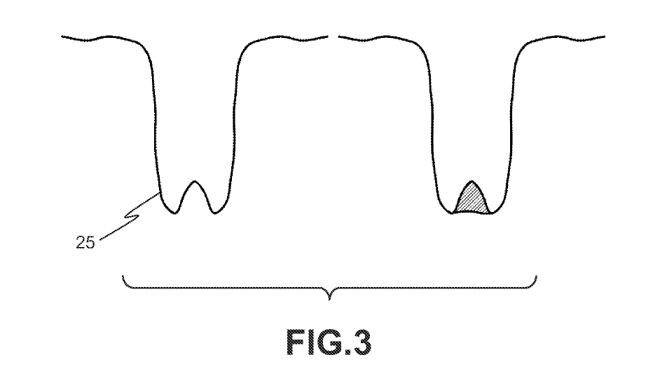

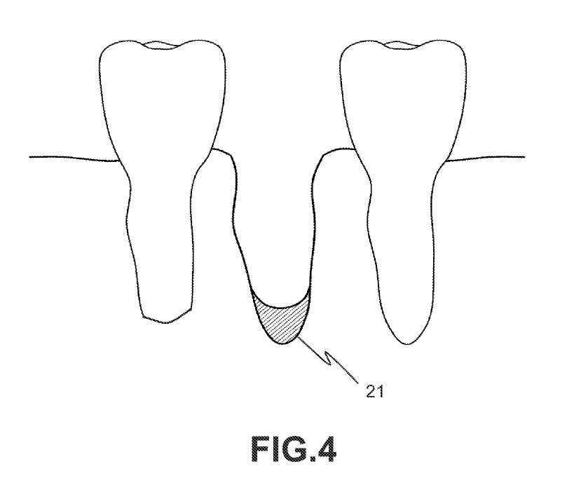

In one embodiment, Triple tray PVS impression method is recommended. A customized bur to remove intra-root bone 25 for any multi-rooted tooth is needed as shown in FIG. 3. An osteotomy to lengthen the existing socket 21 is recommended for a perio involved tooth as show in FIG. 4.

Once the lab receives the impression, the impression is captured via a special 3D dental scanner system into a digital 3D dental CAD software. Using the CAD software, lab technician designs the custom fixture 22 based on the root anatomy, soft tissue of the tooth, tooth location, adjacent teeth location/position, and opposing teeth. The fixture 22 should be designed as an exact negative of the bone socket site with no undercuts and a slightly larger diameter in the abutment end of the fixture 22. Once designed, the software CAD data is sent to a 3D dental CAM milling machine where a titanium block is milled according to the design specification. The implant fixture 22 features most of the same features as the traditional cylindrical fixture 12: grooves to increase surface area with the bones, receiving end for the abutment, screw hole for the abutment screw.

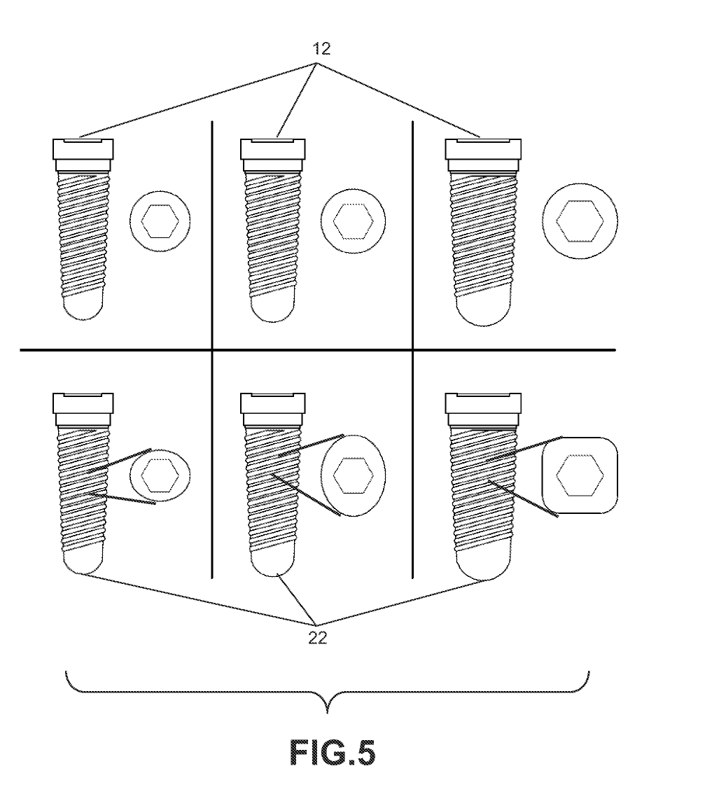

The fundamental difference in design of the custom implant fixture 22 and traditional standardize size implant fixture 12 is that the standardized implant fixture 12 is a screw type implant, that the fixture 12 is screwed into the prepared bone site. The mechanical function of the “screw-like” feature provides the initial stability for the implant fixture 12 to begin osteointegration, whereas, the custom implant fixture 22 functions more like a nail by compressing the bone around the implant which provide the initial stability during the osteointegration period. Thus the geometrical difference between custom implant fixture (CIF) 22 and the traditional implant fixture 12 is that the horizontal cross section area of the custom fixture 22 is not completely round with a constant diameter as in the case of the traditional standardize sized cylindrical implant fixture 12. It can be round for anterior teeth, elliptical for premolars, or rounded rectangle for molars as shown in FIG. 5. Referring to FIG. 5, there is illustrated respectively a perspective view, and a top plan view of the custom implant fixture 22 according to the present invention. The custom implant fixture 22 is designed to conform to the shape of the specific teeth so that it provides a full natural support for the tooth analogue.

Once milled, the fixture 22 surface is coated with osteointegration materials as prescribed by the dentist. Once the custom fixture 22 is fabricated, the lab will also fabricate a custom abutment/implant transfer with the option for a fixture analog depending on the dentist's preference for ways of restoring the implant. Lab tech can design the custom abutment 23 (either metal or ceramic) in the CAD/CAM software based on the same criteria as the custom fixture 22. (soft tissue, location of adjacent and opposing dentition) in addition to the custom fixture data. The custom implant fixture 22, fixture analog (optional) and custom abutment/implant transfer 23 are packaged and mailed back to the dentist office within a week.

At the one-week appointment, the temporary socket maintainer is removed, socket 21 is cleaned with a spoon excavator. Since the custom implant fixture 22 is an exact replica of the extraction/socket site 21 with a slightly larger abutment end, it is tapped or inserted into the socket 21 with minimal force (like a nail) at step 206. Similar to the standard dental implant protocol, a three months period is given to allow the custom implant fixture 22 to osteointegrate with the surrounding bones before the abutment 23 is torqued onto the fixture.

After 3 months, dentist tests for osteointegration of the custom implant fixture 22, then restore the implant abutment 23 at step 208. Similarly to the standard dental implant protocol, an impression is taken for the crown 24. Two weeks later the crown 24 comes back from the lab and is placed onto the abutment 23 at step 210. The entire course takes 3 and ¾ months equivalent to 15 weeks. The dental implant protocol using a custom implant fixture system and method according to the present invention shorts the period from tooth extraction to the completed restoration of the tooth by about 3 months.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A method of performing dental implant using a custom implant fixture to shorten the extraction to completed restoration period, which comprises:
taking an impression after a tooth being extracted to capture the extraction socket, teeth adjacent to the extracted tooth, soft tissue and the opposing dentition and sending the impression to a lab for designing and fabricating a custom implant fixture system including a custom implant fixture and abutment;
filling the extraction socket site with a solid socket space maintainer to prevent the bone from filling in before an implant fixture placement;
removing the temporary socket maintainer and cleaning the socket;
placing the custom implant fixture into the extraction socket;
placing the abutment onto the custom implant fixture; and
placing a crown onto the abutment.

2. The method for performing dental implant of claim 1, which further comprises:
designing and fabricating a custom implant fixture using the information obtained from the impression using CAD/CAM software, wherein the custom implant fixture is designed as an exact negative of the bone socket site and which is a block made with titanium with an apical end and an abutment end and which has grooves or a rough surface on the block to increase contact surface area with bones and a screw hole on the abutment end for receiving an abutment screw and which is coated with osteointegration materials; and
designing and fabricating a custom abutment/implant transfer with the option for a fixture analog in the CAD/CAM software based on the impression information;

3. The method for performing dental implant of claim 2, wherein the custom implant fixture has no undercuts and the abutment end is slightly larger than the apical end.

4. The method for performing dental implant of claim 2, wherein the impression is captured via a special 3D dental scanner system into a digital 3D dental CAD software which enables lab to design specification for the custom fixture based on the root anatomy, soft tissue of the tooth, tooth location, adjacent teeth location/position, and opposing teeth.

5. The method for performing dental implant of claim 4, wherein the custom implant fixture is made from a titanium block and is milled by a 3D dental CAM milling machine according to the design specification which is software CAD data sent to the 3D dental CAM milling machine.

6. The method for performing dental implant of claim 2, wherein the impression is taken using a triple tray PVS impression method.

7. The method for performing dental implant of claim 1, which further comprises using a customized bur to remove intra-root bone for any multi-rooted tooth or performing osteotomy to lengthen the existing socket for a perio-involved tooth.

8. The method for performing dental implant of claim 2, wherein the custom implant fixture is stabilized by compressing the bone around the implant during the osteointegration period.

9. A system for dental implant comprising:
a custom implant fixture; and
a custom abutment/implant transfer;
wherein the custom implant fixture is designed as an exact negative of the bone socket site of an extracted tooth based on an impression and which is a block made of titanium with an apical end and an abutment end and which has grooves on the block to increase surface area with bones and a internally threaded screw hole open to the abutment end for receiving an abutment screw and which is coated with osteointegration materials; and the custom abutment/implant transfer is designed and fabricated based on the impression.

10. The system for dental implant of claim 9, wherein the is designed as an exact negative of the bone socket site of an extracted tooth based on impression using CAD/CAM software and the custom abutment is designed and fabricated using the CAD/CAM software based on the impression information.

11. The system for dental implant of claim 9 further comprising a fixture analog.

12. The system for or dental implant of claim 10, wherein the custom implant fixture is made from a titanium block and is milled by a 3D dental CAM milling machine according to the design specification which is software CAD data sent to the 3D dental CAM milling machine.

13. The system for dental implant of claim 9, wherein the custom implant fixture may have different shapes, including but not limited to round shape, elliptical shape, or rounded rectangle shape.

14. The system for dental implant of claim 9, wherein the custom implant fixture is made from titanium block and the custom abutment is made from titanium or ceramic.

15. A method of designing and fabricating a custom implant fixture and abutment, which comprises:
taking an impression after a tooth being extracted to capture the extraction socket, teeth adjacent to the extracted tooth, soft tissue and the opposing dentition;
designing and fabricating a custom implant fixture using the information obtained from the impression using CAD/CAM software, wherein the custom implant fixture is designed as an exact negative of the bone socket site and which is a block made with titanium with an apical end and an abutment end and which has grooves on the block to increase surface area with bones and a screw hole on the abutment end for receiving an abutment screw and which is coated with osteointegration materials; and
designing and fabricating a custom abutment/implant transfer with the option for a fixture analog in the CAD/CAM software based on the impression information;
wherein the custom implant fixture is stabilized during the osteointegration period by compressing the bone around the implant.

16. The method of designing and fabricating a custom implant fixture and abutment of claim 15, wherein the custom implant fixture has no undercuts and the abutment end is larger than the apical end.

17. The method of designing and fabricating a custom implant fixture and abutment of claim 15, wherein the impression is captured via a special 3D dental scanner system into a digital 3D dental CAD software which enables lab to design specification for the custom fixture based on the root anatomy, soft tissue of the tooth, tooth location, adjacent teeth location/position, and opposing teeth.

18. The method of designing and fabricating a custom implant fixture and abutment of claim 15, wherein the custom implant fixture is made from a titanium block and is milled by a 3D dental CAM milling machine according to the design specification which is software CAD data sent to the 3D dental CAM milling machine.

19. The method of designing and fabricating a custom implant fixture and abutment of claim 15, wherein the impression is taken using triple tray PVS impression method.

20. The method of designing and fabricating a custom implant fixture and abutment of claim 15, wherein the custom implant fixture is stabilized by making abutment end of the implant fixture slightly larger than the socket, so when fixture is fully inserted it compresses the bone around the implant during the osteointegration period.
